(19) United States
(12) Patent Application Publication
(10) Pub. No.: US 2024/0328983 A1
(43) Pub. Date: Oct. 3, 2024

(54) CAPACITANCE BASED LIQUID, ICE, AND FROST SENSOR

(71) Applicant: Regal Beloit America, Inc., Beloit, WI (US)

(72) Inventors: Paul Mullin, Yellow Springs, OH (US); Derrin McCormick, Dayton, OH (US); Connor Silva, Tipp City, OH (US); Joseph Douglas, Sidney, OH (US)

(21) Appl. No.: 18/128,907

(22) Filed: Mar. 30, 2023

(51) Int. Cl. G01N 27/22; G01N 33/18

(52) U.S. Cl. CPC G01N 27/226; G01N 33/18; F25D 21/02; G01N 33/1873

(57) ABSTRACT

A frost sensor, or a frost/ice/water sensor, and systems and methods of using the same, are provided herein. The frost sensor includes a body including a measurement circuit, and a capacitance sensor including two or more co-planar plates coupled to and extending laterally from the body. The measurement circuit is configured to measure a capacitance sensed by the capacitance sensor, the measurement representing a change of material between the plates over time. The frost sensor also includes at least one mounting component coupled to and extending laterally from the body. In some instances, the frost sensor is coupled to evaporator coils of a cooling system.

### BACKGROUND

The field of the disclosure relates generally to a frost sensor, and, more specifically, to a capacitance-based liquid, ice, and frost sensor and systems and method of employing the same.

Cooling systems, such as refrigerators and freezers, are used by entities such as grocery stores and warehouses to store or display foods and beverages at a suitable temperature. Evaporator coils and condenser coils can accumulate foreign bodies thereon, such as ice, dust, and other debris, which decrease the airflow across the components, thereby reducing the efficiency of these coils. Similarly, evaporator and condenser coils in heating, ventilation, and air conditioning (HVAC) systems experience similar debris accumulation.

To detect this ice or dust accumulation, conventional cooling systems can monitor temperature or air flow changes within the cooling system. Additionally or alternatively, cooling systems monitor fan power draw or motor efficiency changes. However, such systems can only detect accumulation after it has reached a critical level, and significant maintenance of the cooling system may be required. In the case of dust or ice accumulation, complete cleaning or de-icing of the coils may be required and can cause further down time of the cooling system. Furthermore, when temperature and air flow have dropped to a critical level, the contents stored within the cooling system have to be assessed and often times may have to be discarded if they have not been maintained at the proper temperature.

In HVAC systems, when frost or debris buildup occurs, reduced heating and cooling capacity is available to satisfy the heating/cooling demand. Efficiency can be significantly reduced, resulting in additional costs for operating the HVAC systems.

Therefore, there exists a need for a system configured to detect early debris accumulation in retail and commercial cooling systems as well in residential and commercial HVAC systems.

### BRIEF DESCRIPTION

In one aspect, a frost sensor is provided. The frost sensor includes a body including a measurement circuit, a capacitance sensor, and at least one mounting component coupled to and extending laterally from the body. The capacitance sensor includes two or more co-planar plates coupled to and extending laterally from the body, wherein the measurement circuit is configured to measure a capacitance sensed by the capacitance sensor, the measurement representing a change of material between the plates over time.

In another aspect, a method of measuring ice buildup in an evaporator coil is provided. The method includes coupling a frost sensor between two adjacent fins of the evaporator coil, the frost sensor including (i) a body including a measurement circuit, (ii) a capacitance sensor including two or more coplanar plates coupled to and extending laterally from the body, and (iii) at least one mounting component coupled to and extending laterally from the body. The method also includes sensing, using the capacitance sensor, a capacitance between the plates, and measuring, by the measurement circuit, the sensed capacitance, the measurement representing a change in material between the plates over time.

In another aspect, a defrost system is provided, the defrost system including a frost senor and an external device. The frost sensor includes a body including a measurement circuit, a capacitance sensor, and at least one mounting component coupled to and extending laterally from the body. The capacitance sensor includes two or more co-planar plates coupled to and extending laterally from the body. The measurement circuit is configured to measure a capacitance sensed by the capacitance sensor, the measurement representing a change in material between the plates over time. The measurement circuit is further configured to transmit the measurement to the external device.

### BRIEF DESCRIPTION OF THE DRAWINGS

FIG. 1 is a block diagram of an example cooling system including an evaporator and a frost sensor;

FIG. 2 is an expanded schematic view of the evaporator shown in FIG. 1 including a frost sensor;

FIG. 3 depicts an example frost sensor;

FIG. 4 illustrates capacitance sensed by the frost sensor over time as frost and ice buildup occurs on evaporator coils;

FIG. 5 is a schematic view of a pipe including a frost sensor coupled to an interior surface thereof;

FIG. 6 is a schematic view of a pipe including a frost sensor coupled to an exterior surface thereof;

FIG. 7 is a block diagram of an example defrost system; and

FIG. 8 is a flow diagram of an example method of measuring ice buildup in an evaporator coil.

### DETAILED DESCRIPTION

The following disclosure provides examples of a frost sensor, as well as systems and methods employing the same. As used herein, “frost sensor” refers broadly to a capacitance-based sensor configured to sense the presence of fluid, liquid (e.g. water), ice, frost, and/or other debris thereon (e.g., based on a sensed capacitance). In one example embodiment, the frost sensor is coupled between fins of an evaporator coil of a cooling system (e.g., a refrigerator cooling system) and is used to measure ice buildup on the evaporator coil. The frost sensor includes a capacitance sensor, such as two or more coplanar plates (e.g., a pair of parallel plates), configured to sense capacitance. The capacitance varies depending on the environment or material between the plates. Specifically, the sensed capacitance is lower when the material between the plates is air and increases when debris accumulates on and between the plates. Although the primary example used in the following disclosure relates to ice—also referred to as frost-buildup on the capacitance sensor, the same applies to any change in material between sensor plates, including buildup of other debris, such as water, dust, and the like. Accordingly, the frost sensor of the present disclosure may be readily applicable to sensing any debris accumulation on coils, on the inside or outside of pipes and/or any other surface that may experience a build-up of debris, across various implementations of cooling and/or HVAC systems.

The frost sensor also includes a measurement circuit that measures the sensed capacitance. This measurement represents any change in material between the plates over time, or, more particularly, the amount of ice buildup between the pair of plates, which serves as a proxy for the ice buildup on the fins between which the frost sensor is positioned. In some embodiments, the measurement circuit compares the measurement to a defrost threshold. The defrost threshold represents a maximum desired or allowed amount of buildup, above which the evaporator coil may exhibit undesirably reduced efficiency.

The frost sensor can be implemented as a component of an overall defrost system, which includes an external device communicatively coupled to the frost sensor. The frost sensor (e.g., the measurement circuit) transmits capacitance measurements to the external device, which can interpret the capacitance measurements to perform remedial actions, such as initiating a defrost cycle within the cooling system. Accordingly, the frost sensor and systems employing the same enable improved frost detection at a low cost, thereby facilitating more timely defrost processes to maintain the efficiency and effectiveness of the components of the overall cooling system.

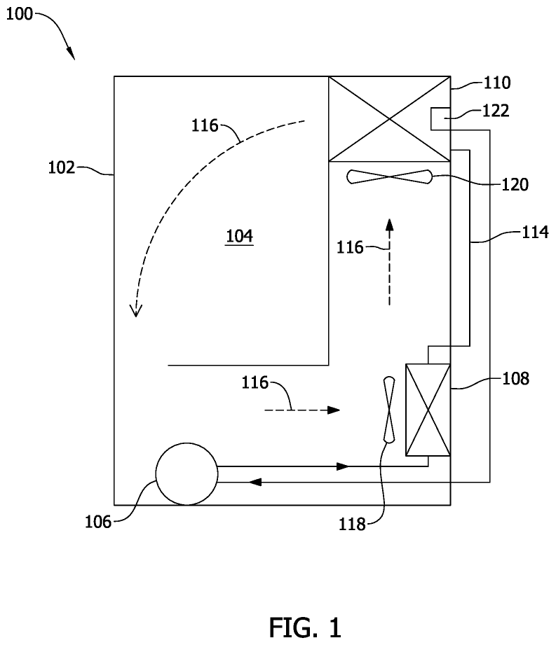

FIG. 1 is a schematic representation of a cooling system 100. Cooling system 100 comprises an enclosure 102 defining an interior space 104, a compressor 106, a condenser 108, and an evaporator 110. In some embodiments, enclosure 102 is a refrigeration cabinet. In some embodiments, interior space 104 has a door or opening though which contents may be inserted, stored, or displayed in enclosure 102. Compressor 106, condenser 108, and evaporator 110 are fluidly connected by refrigeration tubes 114 to form a refrigeration circuit through which refrigerant circulates.

Air 116 is circulated through cooling system 100 to cool contents within interior space 104. Specifically, air 116 is circulated by a condenser fan 118 positioned before condenser 108, and by an evaporator fan 120 positioned before evaporator 110. Condenser fan 118 and evaporator fan 120 are each operated by a respective motor (not shown) to push air 116 into condenser 108 and evaporator 110, respectively. In one example embodiment, cooling system 100 is a low- or medium-temperature refrigeration system. However, the functionality disclosed herein may be applicable in other embodiments, such as in HVAC systems. As described above, in many instances, ice—also referred to as frost—builds up on the coil of evaporator 110 over time. In the example embodiment, cooling system 100 includes a frost sensor 122 configured to sense and measure this ice build-up.

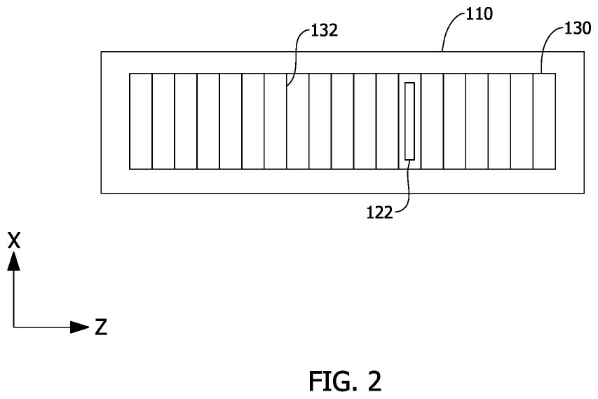

Turning to FIG. 2, an expanded view of evaporator 110 is depicted. Evaporator 110 includes evaporator coil 130 having a plurality of fins 132. In one example embodiment, frost sensor 122 is coupled to two adjacent fins 132, such that frost sensor 122 is positioned between these fins 132. In the example embodiment, sufficient space exists between frost sensor 122 and the adjacent fins 132 to allow airflow between sensor 122 and fins 132. Therefore, the accumulation of debris (e.g., frost) on frost sensor 122 is a sufficiently accurate proxy for debris accumulation on fins 132. In one example embodiment, a majority of frost sensor 122 is located between the adjacent fins 132 when frost sensor 122 is coupled to evaporator coil 130. Frost sensor 122 may therefore be understood as being surrounded by fins 132.

In some embodiments, multiple frost sensors 122 are used in cooling system 100. For example, two or more sensors 122 may be coupled to evaporator coil 130 at different locations, to compensate for uneven frost buildup. Additionally or alternatively, for each motor that drives a fan in cooling system 100, a corresponding frost sensor 122 may be employed to detect debris buildup on the respective component.

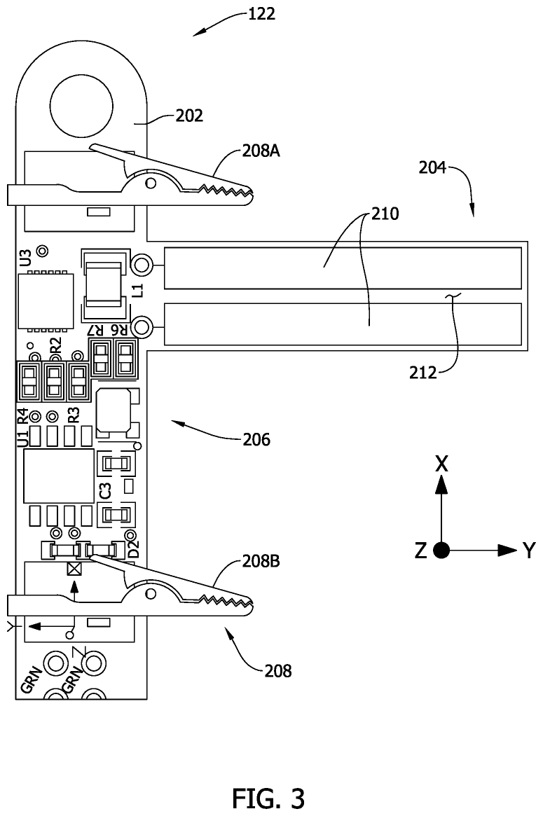

FIG. 3 depicts one example embodiment of frost sensor 122. Frost sensor 122 includes a body 202 that extends longitudinally or lengthwise in a longitudinal direction X. Body 202 functions as a structural support for various other components of frost sensor 122, including a capacitance sensor 204, a measurement circuit 206, and mounting components 208. Body 202 may be constructed from any rigid material, including polymeric materials, metallic materials, and/or combinations thereof. In particular, non-conductive rigid materials are used, such that the material of body 202 does not interfere with capacitance measurements of frost sensor 122. In some embodiments, body 202 is formed from a resin and fiberglass composite material (e.g., FR-4).

In one example embodiment, frost sensor 122 is designed to be coupled to fins (e.g., fins 132) of an evaporator coil (e.g., coil 130). In such an embodiment, frost sensor 122 has dimensions suitable for frost sensor 122 to be positioned between fins 132 while avoiding interference with any other component of coil 130 (e.g., internal piping). Frost sensor 122 may accordingly have a length, measured in the longitudinal direction X, of about 40-60 millimeters (mm), or about 50 mm, and a width (e.g., of body 202), measured in a lateral direction Y, of about 7-15 mm, or about 9-10 mm. Frost sensor 122 may also have a thickness, measured in a transverse direction Z, of about 12 mm or 0.45 inches (in). Where frost sensor 122 is employed in other applications, frost sensor 122 may have other suitable dimensions.

Mounting components 208, illustrated as clips in the example embodiment of FIG. 3, are configured to couple frost sensor 122 to fins 132. In particular, mounting components 208 are arranged such that frost sensor 122 is coupled between two adjacent fins 132 and is held stably in place. Reducing or eliminating movement of frost sensor 122, such as when air (e.g., air 116) is circulated across coil 130 and frost sensor 122, facilitates improving the accuracy and reliability of capacitance measurements.

In one embodiment, frost sensor 122 includes two mounting components 208 coupled to body 202. Mounting components 208 extend outwardly from body 202, laterally in a lateral direction Y. One mounting component 208A is coupled to body 202 above capacitance sensor 204 with respect to direction X, or adjacent a first side of capacitance sensor 204. A second mounting component 208B is spaced from the first mounting component 208A in direction X, and is below capacitance sensor 204 or adjacent a second, opposing side of capacitance sensor 204. Mounting components 208 may be coupled to body 202 on opposing sides thereof (e.g., on transversely opposing sides of body 202), such that one mounting component 208 is closer to one fin 132 and the other mounting component 208 is closer to the adjacent fin 132 when frost sensor 122 is positioned between fins 132.

Mounting components 208 are configured to hold frost sensor 122 still and to maintain a consistent, known distance between frost sensor 122 (e.g., plates 210, described below) and fins 132. In some embodiments, frost sensor 122 additionally includes one or more centering or spacing components, which function as part of or in conjunction with mounting components 208 to maintain the intended spacing between fins 132 and frost sensor 122. For example, frost sensor 122 includes spacing components implemented as overmolded spacers (e.g., pins) to center frost sensor 122 between adjacent fins 132 (or otherwise maintain a known, intended spacing between sensor 122 and fins 132). Although mounting components 208 are depicted as clips (e.g., alligator clips) in the illustrated embodiment, mounting components 208 may be implemented in a variety of suitable ways, and the size, number, type, and orientation of mounting components 208 may vary. For example, in some cases, mounting screws may be implemented, or frost sensor 122 may be friction-fit in its desired position.

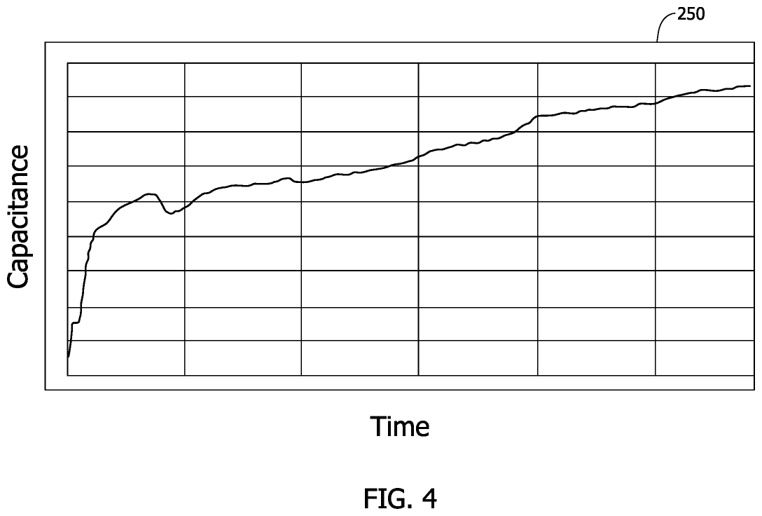

Capacitance sensor 204 includes two or more coplanar plates 210 having a spacing 212 therebetween. In the illustrated embodiment of FIG. 3, co-planar plates 210 are specifically embodied as two parallel plates 210. Capacitance sensor 204 senses the capacitance between plates 210. This capacitance varies depending upon the material disposed within spacing 212, such as air, water, ice, dust, and the like. The capacitance increases over time as debris builds up on plates 210, as shown in the graph 250 depicted in FIG. 4. Therefore, the sensed capacitance between plates 210 can be used to generate a suitably accurate proxy measurement for the buildup of debris on fins 132.

Plates 210 extend laterally from body 202 in the lateral direction Y. Therefore, when frost sensor 122 is coupled to fins 132, plates 210 extend at least in part in a same plane as, or parallel to, fins 132. In the example embodiment, plates 210 are co-planar and are parallel to one another, spaced closed together to concentrate the sensing area. In other embodiments, plates 210 may be other than parallel or other than co-planar. Increasing the distance between plates 210 may increase the sensing range of frost sensor 122. Plates 210 have dimensions suitable for the particular application of frost sensor 122. For example, where frost sensor 122 is coupled between adjacent fins 132 of a 7 FPI (fins per inch) evaporator coil 130, each plate 210 may have a length in the longitudinal direction X of about 5-10 mm, or about 7.5 mm, and a width in the lateral direction Y of about 15-30 mm, or about 25-30 mm. Spacing 212 between plates 210 may be less than 1 mm in some embodiments, or less than 0.5 mm.

More generally, depending on the particular application, plates 210 can be very small (e.g., less than 1 mm²) or very large (e.g., hundreds of mm²). Smaller plates 210 enable installation in evaporator coils, as described herein. Larger plates 210 may be employed to increase an area across which capacitance is being measured, which can be advantageous where the precise location of initialization of frost/ice buildup is unknown. Therefore, the specific dimensions and relative spacing of plates 210 may be selected based upon the sensing requirements of the particular application of frost sensor 122.

In alternative embodiments, plates 210 are other than co-planar. For example, plates 210 may be flexible plates configured to wrap around a component for sensing frost buildup therearound. In other embodiments, capacitance sensor 204 includes more than two plates 210. For example, capacitance sensor 204 may include three or more plates 210 that are wired in parallel. In some such cases, these parallel-wired plates 210 may be arranged to effectively form a larger plate 210, to increase or change the sensing area of capacitance sensor 204.

Measurement circuit 206 is coupled to body 202, and is configured to measure the capacitance sensed by capacitance sensor 204, in Farads (F). Measurement circuit 206 is configured to generate capacitance measurements substantially continuously. In other embodiments, measurement circuit 206 generates capacitance measurements periodically. The particular periodicity of measurements may vary depending on the application of frost sensor 122, and should be frequent enough that capacitance changes are readily detectable. It may be desirable to minimize measurements (or the transmission of measurements, as described herein) to optimize system efficiency. In some embodiments, in which ice buildup is relatively slow (e.g. over the course of days), hourly measurements may be sufficient to track buildup over time, before a threshold is reached. In other embodiments, ice buildup is relatively fast, and capacitance measurements may be generated and/or transmitted multiple times per hour. For example, in some commercial refrigeration units, measurement circuit 206 may measure capacitance 4-6 times per hour (or transmit capacitance measurements 4-6 times per hour)—or once per 50 ms, or once per minute, up to an indefinite amount of time between measurements—such that any failure event (e.g. a door being left open or a defrost heater fails) is detectable.

In one example embodiment, a calibration of frost sensor 122 may be implemented over a period of time. In this calibration phase, various baseline or standard parameters may be determined, such as a baseline buildup of debris, a baseline rate of buildup, a baseline defrost schedule, and the like. The periodicity of capacitance measurements and/or transmissions thereof may be selected or tuned based on the outcome of this calibration phase.

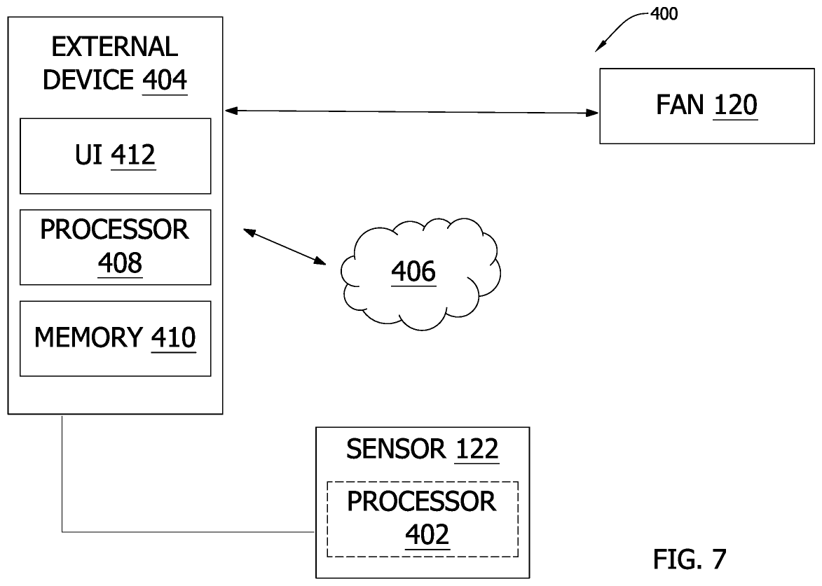

In some embodiments, frost sensor 122 also includes a processing component (e.g., processing component or processor 402, shown in FIG. 7), which enables various computations to be made locally at frost sensor 122. The processing component may be embodied as part of measurement circuit 206. The processing component may, for example, compare capacitance measurements to one or more stored threshold values. Measurement circuit 206 may include or be communicatively coupled to a communication interface (not shown), which enables wired and/or wireless communication of measurements and/or other data from frost sensor 122 to external device(s), as described further herein. Specifically, in the example embodiments, processing of data from measurement circuit 206 is performed at an external device (e.g., a cloud-based computation device).

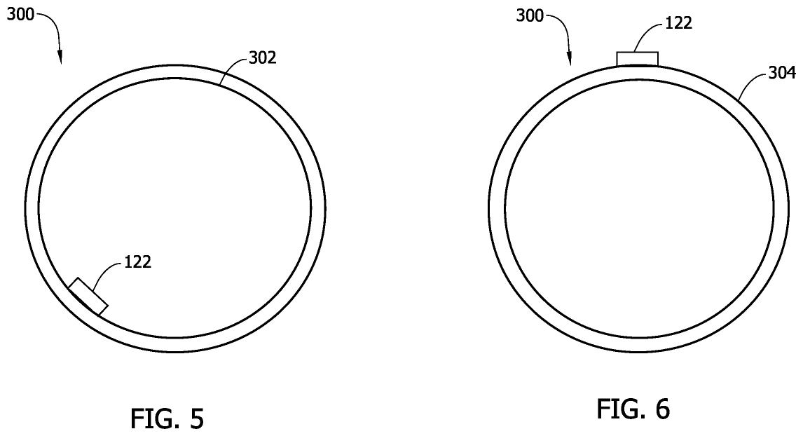

Turning to FIGS. 5 and 6, alternative implementations of frost sensor 122 is depicted, in which frost sensor 122 senses frost (or water, ice, debris, etc.) buildup relative to a pipe 300. In particular, FIG. 5 depicts frost sensor 122 coupled to an interior surface 302 of pipe 300, and FIG. 6 depicts frost sensor 122 coupled to an exterior surface 304 of pipe 300. Pipe 300 can be any pipe or piping component of a larger system, such as cooling system 100, defrost system 400 (shown in FIG. 7), an HVAC system, or any other system in which fluid (e.g., liquid or gas) is transported between components via pipes. The relative size of frost sensor 122 relative to pipe 300 shown in FIGS. 5 and 6 should not be considered limiting or to scale; rather, the size of frost sensor 122 relative to pipe 300 can be selected based upon the particular implementation parameters of the system in which frost sensor 122 is applied.

With respect to FIG. 5, frost sensor 122 is configured to sense frost buildup using capacitance measurements as described elsewhere herein, relative to interior surface 302 of pipe 300. Excess ice or debris buildup on the inside of pipes can lead to blockages or clogs, especially in outdoor piping (e.g., piping used for sprinkler systems and pools). As also described herein, frost sensor 122 may transmit the capacitance measurements to an external device configured to monitor the measurements relative to a threshold value, such as a threshold capacitance value representative of a threshold amount of buildup on interior surface 302. When the threshold value is reached, the external device is configured to transmit a signal including an alert to a client computing device of a user. The alert indicates to the user that a threshold or critical amount of ice (or other debris) has built up on interior surface 302 and a remedial action is needed.

With respect to FIG. 6, frost sensor 122 is configured to sense frost buildup using capacitance measurements as described elsewhere herein, relative to exterior surface 304 of pipe 300. Excess ice on the outside of piping can pose a risk, in cases where a large amount of ice builds up and subsequently melts, which can overwhelm the system's ability to process liquid water. As also described herein, frost sensor 122 may transmit the capacitance measurements to an external device configured to monitor the measurements relative to a threshold value, such as a threshold capacitance value representative of a threshold amount of buildup on exterior surface 304. When the threshold value is reached, the external device is configured to transmit a signal including an alert to a client computing device of a user. The alert indicates to the user that a threshold or critical amount of ice (or other debris) has built up on exterior surface 304 and a remedial action is needed.

With reference now to FIG. 7, frost sensor 122 (as a component of cooling system 100) functions as a component of a defrost system 400 configured to facilitate efficient defrosting (or other remedial action) of cooling system 100. Defrost system 400 includes one or more frost sensors 122, as described herein, as well as an external device 404.

External device 404 is communicatively coupled to frost sensor(s) 122 via a communication network 406, which may include a wired communication network (e.g., Ethernet, MODBUS, BACNET) and/or wireless communication network (e.g., a radio communication network, BLUETOOTH, ZIGBEE, LAN, etc.). External device 404 includes a processor 408, a memory 410, and a user interface (UI) 412. External device 404 may include any computing device, such a server computing device or user computing device (e.g., laptop, desktop, smartphone, tablet, etc.). Moreover, in some embodiments, defrost system 400 includes more than one external device 404.

In one example embodiment, frost sensor 122 transmits capacitance measurements and/or other data from frost sensor 122 to external device 404. External device 404 may be configured to process and store any received data (e.g., via processor 408 and memory 410). In some embodiments, frost sensor 122 (e.g., via processor 402) is configured to perform one or more preliminary computations or calculations using the capacitance measurements, and transmits the results of those processes to external device 404. Additionally or alternatively, frost sensor 122 transmits raw capacitance measurement data to external device 404, where any computations are performed. The results of any computations or analyses may be displayed to a user (not shown) via UI 412. UI 412 may be, for example, a web page or app page. For example, UI 412 may be generated using one or more of an ANDROID or iOS phone app or an AZURE web app.

In the example embodiment, a defrost threshold value is stored, and the capacitance measurement is continuously or periodically compared to this defrost threshold value. As described herein, this comparison may be performed locally at frost sensor 122 or at external device 404. The defrost threshold value represents an amount of frost buildup at which evaporator 110 may operate at an undesirable efficiency. When this defrost threshold value is met or exceeded, one or more remedial actions may be initiated. For example, a signal may be sent to evaporator fan 120 (or a motor operating fan 120) to increase an airflow output from evaporator fan 120, which may reduce the amount of ice or other debris on fins 132 (and sensor 122). Other remedial actions may include instructions for manual intervention (e.g., manual cleaning or defrosting of evaporator coil 130), or the scheduling of a defrost cycle at a future time. In some embodiments, more than one defrost threshold value may be stored, and different actions are initiated when different thresholds are met. For instance, when a first (lower) threshold is met, an alert may be generated by external device 404 for display to a user thereof, whereas at a second (higher) threshold, a defrost cycle may be initiated.

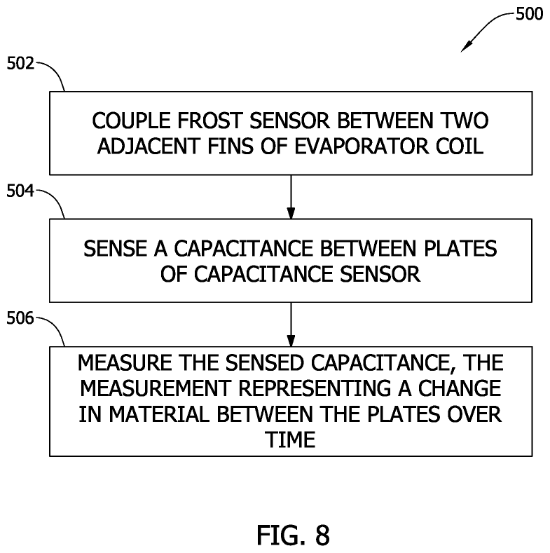

FIG. 8 is a flow diagram of a method 500 of measuring ice buildup in an evaporator coil (e.g., evaporator coil 130, shown in FIG. 2). Method 500 may be implemented using one or more components of cooling system 100 and/or defrost system 400 (shown in FIG. 7), such as frost sensor 122 and/or external device 404.

Method 500 includes coupling 502 a frost sensor (e.g., frost sensor 122) between two adjacent fins of the evaporator coil. As described herein, the frost sensor includes (i) a body including a measurement circuit, (ii) a capacitance sensor comprising two or more co-planar plates coupled to and extending laterally from the body, and (iii) at least one mounting clip coupled to and extending laterally from the body. Method 500 also includes sensing 504, using the capacitance sensor, a capacitance between the plates, and measuring 506, by the measurement circuit, the sensed capacitance, the measurement representing an amount of ice buildup between the plates.

Method 500 may include additional and/or alternative steps, including those described elsewhere herein. For example, in some embodiments, coupling 502 includes coupling 502 the at least one mounting component to a corresponding at least one fin of the two adjacent fins of the evaporator coil. In some embodiments, the at least one mounting component includes two mounting clips, and coupling 502 includes coupling 502 the two mounting clips to, respectively, the two adjacent fins of the evaporator coil.

In some embodiments, method 500 may include transmitting, by the measurement circuit, the measurement to an external device. In some embodiments, measuring 506 includes measuring 506 the sensed capacitance continuously. In other embodiments, measuring 506 includes measuring 506 the sensed capacitance periodically.

In the foregoing specification and the claims that follow, a number of terms are referenced that have the following meanings.

As used herein, an element or step recited in the singular and preceded with the word “a” or “an” should be understood as not excluding plural elements or steps, unless such exclusion is explicitly recited. Furthermore, references to “example implementation” or “one implementation” of the present disclosure are not intended to be interpreted as excluding the existence of additional implementations that also incorporate the recited features.

“Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here, and throughout the specification and claims, range limitations may be combined or interchanged. Such ranges are identified and include all the sub-ranges contained therein unless context or language indicates otherwise.

Disjunctive language such as the phrase “at least one of X, Y, or Z,” unless specifically stated otherwise, is generally understood within the context as used to state that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present. Additionally, conjunctive language such as the phrase “at least one of X, Y, and Z,” unless specifically stated otherwise, should also be understood to mean X, Y, Z, or any combination thereof, including “X, Y, and/or Z.”

Some embodiments involve the use of one or more electronic processing or computing devices. As used herein, the terms “processor” and “computer” and related terms, e.g., “processing device,” “computing device,” and “controller” are not limited to just those integrated circuits referred to in the art as a computer, but broadly refers to a processor, a processing device, a controller, a general purpose central processing unit (CPU), a graphics processing unit (GPU), a microcontroller, a microcomputer, a programmable logic controller (PLC), a reduced instruction set computer (RISC) processor, a field programmable gate array (FPGA), a digital signal processing (DSP) device, an application specific integrated circuit (ASIC), and other programmable circuits or processing devices capable of executing the functions described herein, and these terms are used interchangeably herein. The above embodiments are examples only, and thus are not intended to limit in any way the definition or meaning of the terms processor, processing device, and related terms.

In the embodiments described herein, memory may include, but is not limited to, a non-transitory computer-readable medium, such as flash memory, a random access memory (RAM), read-only memory (ROM), erasable programmable read-only memory (EPROM), electrically erasable programmable read-only memory (EEPROM), and non-volatile RAM (NVRAM). As used herein, the term “non-transitory computer-readable media” is intended to be representative of any tangible, computer-readable media, including, without limitation, non-transitory computer storage devices, including, without limitation, volatile and non-volatile media, and removable and non-removable media such as a firmware, physical and virtual storage, CD-ROMs, DVDs, and any other digital source such as a network or the Internet, as well as yet to be developed digital means, with the sole exception being a transitory, propagating signal. Alternatively, a floppy disk, a compact disc-read only memory (CD-ROM), a magneto-optical disk (MOD), a digital versatile disc (DVD), or any other computer-based device implemented in any method or technology for short-term and long-term storage of information, such as, computer-readable instructions, data structures, program modules and sub-modules, or other data may also be used. Therefore, the methods described herein may be encoded as executable instructions, e.g., “software” and “firmware,” embodied in a non-transitory computer-readable medium. Further, as used herein, the terms “software” and “firmware” are interchangeable, and include any computer program stored in memory for execution by personal computers, workstations, clients and servers. Such instructions, when executed by a processor, cause the processor to perform at least a portion of the methods described herein.

Also, in the embodiments described herein, additional input channels may be, but are not limited to, computer peripherals associated with an operator interface such as a mouse and a keyboard. Alternatively, other computer peripherals may also be used that may include, for example, but not be limited to, a scanner. Furthermore, in the exemplary embodiment, additional output channels may include, but not be limited to, an operator interface monitor.

The systems and methods described herein are not limited to the specific embodiments described herein, but rather, components of the systems and/or steps of the methods may be utilized independently and separately from other components and/or steps described herein.

Although specific features of various embodiments of the disclosure may be shown in some drawings and not in others, this is for convenience only. In accordance with the principles of the disclosure, any feature of a drawing may be referenced and/or claimed in combination with any feature of any other drawing.

This written description uses examples to provide details on the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A frost sensor comprising:
a body comprising a measurement circuit;
a capacitance sensor comprising two or more co-planar plates coupled to and extending laterally from the body, wherein the measurement circuit is configured to measure a capacitance sensed by the capacitance sensor, the measurement representing a change of material between the plates over time; and
at least one mounting component coupled to and extending laterally from the body.

2. The frost sensor of claim 1, wherein the measurement represents the change of material including a buildup of ice between the plates over time.

3. The frost sensor of claim 1, wherein the plates extend laterally from the body in a first direction, and the at least one mounting component extending laterally from the body in the same first direction.

4. The frost sensor of claim 1, wherein the body has a thickness of 0.45 inches (in) or less.

5. The frost sensor of claim 1, wherein the at least one mounting component comprises a first mounting clip and a second mounting clip.

6. The frost sensor of claim 5, wherein the second mounting clip is spaced from the first mounting clip in a longitudinal direction.

7. The frost sensor of claim 5, wherein the first mounting clip is coupled to the body adjacent a first side of the capacitance sensor and the second mounting clip is coupled to the body adjacent a second, opposing side of the capacitance sensor.

8. The frost sensor of claim 1, wherein the measurement circuit is further configured to transmit the measurement to an external device.

9. The frost sensor of claim 1, wherein the measurement circuit is configured to measure the sensed capacitance continuously.

10. The frost sensor of claim 1, wherein the measurement circuit is configured to measure the sensed capacitance periodically.

11. A method of measuring ice buildup in an evaporator coil, the method comprising:
coupling a frost sensor between two adjacent fins of the evaporator coil, the frost sensor including (i) a body including a measurement circuit, (ii) a capacitance sensor including two or more coplanar plates coupled to and extending laterally from the body, and (iii) at least one mounting component coupled to and extending laterally from the body;
sensing, using the capacitance sensor, a capacitance between the plates; and
measuring, by the measurement circuit, the sensed capacitance, the measurement representing a change in material between the plates over time.

12. The method of claim 11, wherein the coupling comprises coupling the at least one mounting component to a corresponding at least one fin of the two adjacent fins of the evaporator coil.

13. The method of claim 11, wherein the at least one mounting component includes two mounting clips, and wherein the coupling comprises coupling the two mounting clips to, respectively, the two adjacent fins of the evaporator coil.

14. The method of claim 11, further comprising transmitting, by the measurement circuit, the measurement to an external device.

15. The method of claim 11, wherein the measuring comprises measuring the sensed capacitance continuously.

16. The method of claim 11, wherein the measuring comprises measuring the sensed capacitance periodically.

17. A defrost system comprising:
a frost sensor comprising:
a body comprising a measurement circuit;
a capacitance sensor comprising two or more co-planar plates coupled to and extending laterally from the body, wherein the measurement circuit is configured to measure a capacitance sensed by the capacitance sensor, the measurement representing a change in material between the plates over time; and
at least one mounting component coupled to and extending laterally from the body; and
an external device, wherein the measurement circuit is further configured to transmit the measurement to the external device.

18. The defrost system of claim 17, wherein the external device is configured to process the measurement received from the frost sensor.

19. The defrost system of claim 17, wherein the measurement circuit is configured to transmit the measurement to the external device periodically.

20. The defrost system of claim 17, wherein the measurement circuit is configured to transmit the measurement to the external device at a maximum rate of once per minute.
